(19) United States
(12) Patent Application Publication
(10) Pub. No.: US 2018/0188165 A1
(43) Pub. Date: Jul. 5, 2018

(54) NITROGEN DIOXIDE SENSOR

(71) Applicant: Veris Industries, LLC, Tualatin, OR (US)

(72) Inventor: John H. SHELEY, Portland, OR (US)

(21) Appl. No.: 15/788,096

(22) Filed: Oct. 19, 2017

Related U.S. Application Data
(60) Provisional application No. 62/440,577, filed on Dec. 30, 2016.

(51) Int. Cl. G01N 21/39; G01N 21/31; G01N 21/01; G01N 33/00; H01L 31/167

(52) U.S. Cl. CPC G01N 21/39; G01N 21/31; G01N 21/01; G01N 2201/0639; H01L 31/167; G01N 2021/399; G01N 2201/0612; G01N 33/0037

(57) ABSTRACT

A sensor that preferably senses NO2.

### CROSS-REFERENCE TO RELATED APPLICATIONS

This application claims the benefit of U.S. Provisional Patent Application No. 62/440,577, filed Dec. 30, 2016.

### BACKGROUND OF THE INVENTION

The present invention relates to a nitrogen dioxide sensor.

Nitrogen dioxide is a reddish brown gas at room temperatures that has a pungent acrid odor. Nitrogen dioxide is naturally occurring in the environment, such as from the stratosphere, bacterial respiration, where it absorbs sunlight and regulates the chemistry of the troposphere. While nitrogen dioxide is naturally occurring, it is also a byproduct of chemical processes, such as an internal combustion engine burning fossil fuels and other industrial processes. Prolonged exposure to nitrogen dioxide increases a risk of occupational lung diseases and high exposure to nitrogen dioxide can cause death. In general, it is desirable to have a sensor to detect nitrogen dioxide in different environments and provide an alarm condition when the level of nitrogen dioxide exceeds a threshold level.

To provide for environmental science and air quality control the nitrogen dioxide sensor should detect trace gases in the parts-per-million (ppm) and/or parts-per-billion (ppb or 10⁹) and/or parts-per-trillion (ppt or 10¹²) levels. The nitrogen dioxide sensors may be based upon chemical conversion technology. The chemical conversion based sensor technology consumes the target gas species to generate a measurable signal, which inherently, distorts the desired measurement through the consumption of the gas. Further, the consumption of the NO₂ gas results in an accumulation of waste products that degrade the performance of the chemical conversion based sensor over time. Further, solvents used in chemical conversion based sensors tend to evaporate. The evaporation of reaction solvents eventually degrades the accuracy performance of the reported gas concentration.

Other nitrogen dioxide sensors may include laser-based techniques because of their ability to provide real-time monitoring capabilities with a high degree of sensitivity and selectivity. A NO₂ sensor capable of high sensitivity and selectivity can monitor atmospheric air quality as well as real-time study of the complex photochemical reactions that the NO₂ gases undergo in the atmosphere. Different spectroscopic techniques have been developed for trace gas detection. Spectroscopic techniques that are commonly employed include, absorption spectroscopy using long pass absorption cells such as multipass and Herriott cells, optical cavity methods, photo-acoustic and quartz-enhanced photo-acoustic spectroscopy, and Faraday rotation spectroscopy. Different data processing and analysis procedures have been applied such as frequency modulated spectroscopy techniques to improve the signal to noise ratio and multiple line integrated absorption spectroscopy to improve the sensitivity of detection. Unfortunately, such sensors do not tend to be robust and are complex.

It is desirable to have an optical based sensor that is robust.

The foregoing and other objectives, features, and advantages of the invention will be more readily understood upon consideration of the following detailed description of the invention, taken in conjunction with the accompanying drawings.

### BRIEF DESCRIPTION OF THE SEVERAL VIEWS OF THE DRAWINGS

FIG. 1 illustrates a laser diode assembly.

FIG. 2 illustrates a gas sensor assembly.

FIG. 3 illustrates another gas sensor assembly.

FIG. 4 illustrates a conde from a laser light assembly.

FIG. 5 illustrates an absorption of a silicon junction sensor.

FIG. 6 illustrates an exemplary gas sensor.

### DETAILED DESCRIPTION OF PREFERRED EMBODIMENT

NO₂ has an absorption spectra in the ultraviolet and in the visible regions covering approximately 250-800 nm. The absorption spectra in this region are relatively high with a broad peak in the 350-450 nm range and a large number of intense sub-peaks extending over the 350-450 nm region. This region of the absorption spectra contains a strong, dense spectrum that is well suited for use in the trace detection of NO₂ using an optical technique. High power diode lasers are currently available at 405±10 nm which covers a portion of this absorption spectra.

While trying to detect NO₂ there can be other atmospheric elements and trace gasses that may impact the ability to differentiate the NO₂. For example, some elements that have a potential for interference include H₂O, O₃, SO₂, and NO₃. While the absorption spectrum of each of these elements is relatively broad, the absorption near the range of 405 nm is minimal. Accordingly, using a wavelength in the 405 nm range, such as generally 405±10 nm reduces the interference posed by other types of likely elements. The NO₂ concentrations are typically between 1 part per million to 10 parts per million.

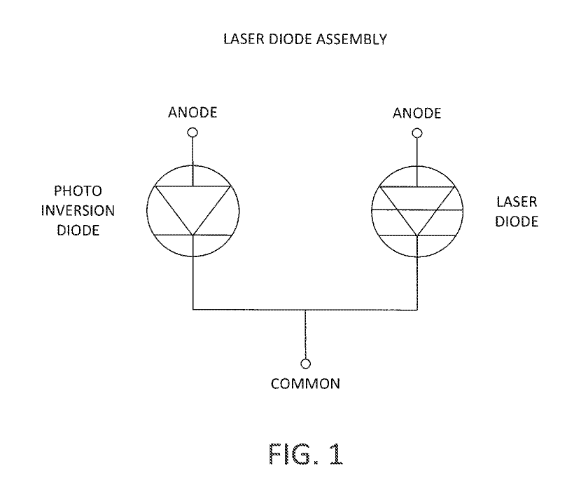

Referring to FIG. 1, a laser diode assembly may include a laser diode that provides an output that includes the 405±10 nm wavelength. Preferably, the output is 75% or more in the 405±10 nm wavelength and more preferably 90% or more in the 405±10 nm wavelength. The laser diode assembly may include a laser diode that provides the 405±10 nm output at an output anode. With the variable nature of the temperature for different environments expected to be encountered for the NO₂ sensor, it is desirable that the laser diode assembly includes a photo inversion diode that senses at an anode the output from the laser diode to provide feedback to the laser diode to control its output so that it is temperature stabilized. The laser diode assembly may also include a common voltage input. In this manner, the laser diode assembly includes stabilizing control feedback in response to thermal changes. Further the feedback may be used to maintain the diode output spectrum, namely, the emitted power versus wavelength, to be maintained substantially stable. Moreover, typically the volume required to be irradiated is between 0.5 liters to 1.2 liters of gas for detectors between 1 part per million and 10 parts per million to sense an optical signal with a sufficient signal to noise ratio.

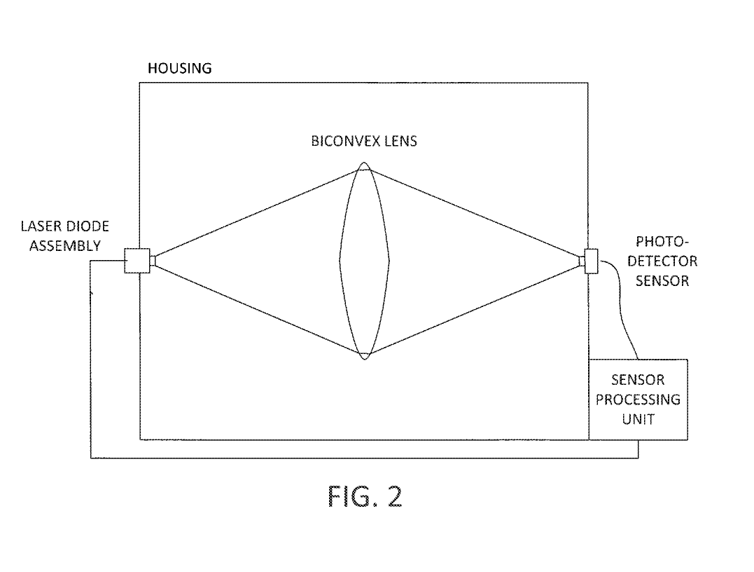

Referring to FIG. 2, a NO₂ sensor may include a substantially enclosed housing that includes one or more openings therein, so that the environmental conditions present within the area proximate the NO₂ sensor housing may be sensed. The NO₂ sensor may include a laser diode assembly affixed to one side of the housing that provides the 405±10 nm optical light output into the enclosure. The laser diode assembly may be supported by the housing in any suitable manner, such as inside and/or outside the housing. The light from the laser diode assembly may result in an increasing cone of light. The cone of light may pass through a biconvex lens which focuses the cone of light from the laser diode assembly on a photodetector sensor, such as a pin diode sensor, affixed to the opposing side of the housing. The photodetector sensor may be supported by the housing in any suitable manner, such as inside and/or outside the housing. A sensor processing unit receives an output from the photodetector sensor over time and based upon a change in the output from the photodetector sensor, such as an attenuated signal, estimates the levels of NO₂ within the housing. The sensor processing unit may provide control signals to the laser diode assembly. The sensor processing unit may be supported by the housing in any suitable manner, such as inside and/or outside the housing. While the NO₂ sensor illustrated in FIG. 1 is functional, any misalignment of the laser diode assembly during assembly or subsequent use will result in the light not being suitably focused on the photodetector sensor. Also, any misalignment of the biconvex lens during assembly or subsequent use will result in the light not being suitably focused on the photodetector sensor. Further, any misalignment of the photodetector sensor during assembly or subsequent use will result in the light not being suitably focused on the photodetector sensor. Moreover, to obtain a sufficient attenuation in the optical light through the housing as a result of relatively low levels of NO₂ gasses, it is desirable to have a substantial volume within the housing, which requires a relatively large housing which can be inconvenient for many applications.

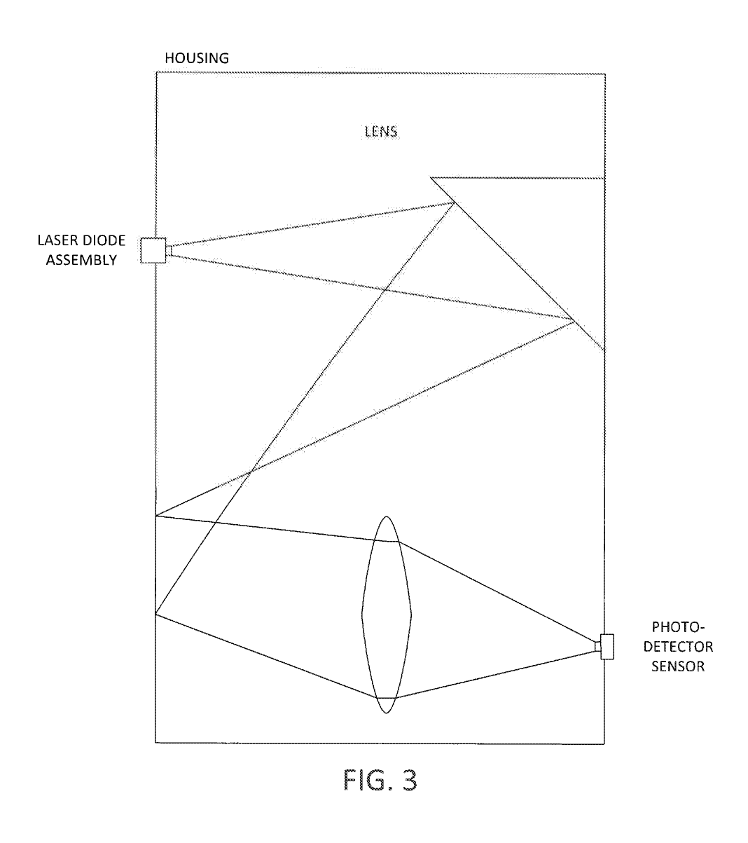

Referring to FIG. 3, another embodiment includes a NO₂ sensor that includes a housing that includes one or more openings therein. The NO₂ sensor may include a laser diode assembly affixed to one side of the housing that provides the 405+10 nm optical light output into the enclosure. The light from the laser diode assembly may result in an increasing cone of light. The cone of light may pass through a set of one or more different lens and one or more reflecting surfaces which ultimately focuses the cone of light from the laser diode assembly on a photodetector sensor, such as a pin diode sensor, affixed to the side of the housing. A sensor processing unit receives an output from the photodetector sensor over time and based upon a change in the output from the photodetector sensor, such as an attenuated signal, determines the levels of NO₂ within the housing. The sensor processing unit may provide control signals to the laser diode assembly. While the NO₂ sensor illustrated in FIG. 3 is functional, any misalignment of the laser diode assembly, the one or more lenses, the one or more reflecting surfaces, and/or the photodetector sensor will result in the light not being suitably focused on the photodetector sensor. With an ever increasing number of components included in the path of the cone of light from the laser diode assembly, the tendency for optical misalignment of one or more of the components increases.

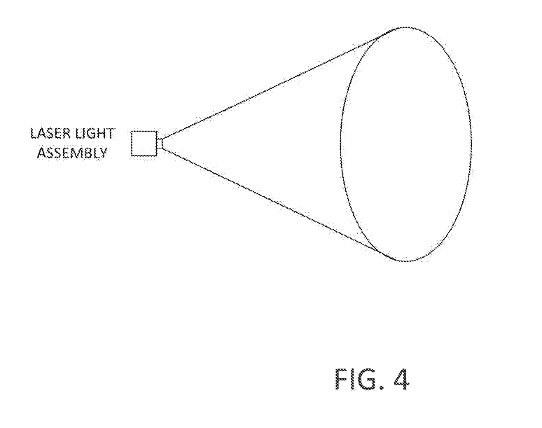

Referring to FIG. 4, the light from the laser diode assembly preferably has an oval elliptical perpendicular cross sectional shape. For example, the radiation deviation in degrees may be substantially 8 degrees from parallel and 21 degrees from perpendicular.

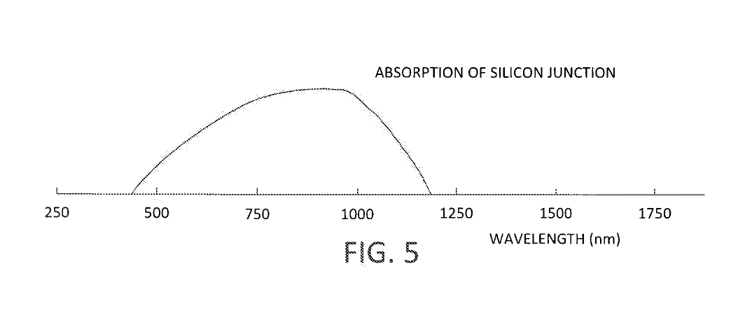

Referring to FIG. 5, in contrast to attempting to further modify the path of the light to maintain sufficiently controlled tolerances, it was determined that a large area photodetector would be preferable as a sensor, since the light reaching the large area photodetector may have substantial variation in its physical location while still being effectively sensed. Moreover, the photodetector should have insubstantial changes in its surface area based upon changes in the ambient temperature, such as between −5 degrees C. to 35 degrees C., with changes in the ambient pressure between 97 kilopascals to 103 kilopascals. A preferred sensor is a solar cell, such as single junction silicon photovoltaic cells. In general the absorption of a silicon junction photovoltaic cell has a maximum absorption around a wavelength of generally 1000 nm. While such a solar cell would not be generally considered an appropriate structure for a detector for a laser diode assembly having an output in the range of 405±10 nm light output, the tail of the absorption of the photovoltaic cell extends into the 300 nm range, and accordingly includes the range of 405±10 nm, albeit with relatively low relative sensitivity. By way of example, the solar cell may have an area of 3 square inches or more, and more preferably an area of 6 square inches or more.

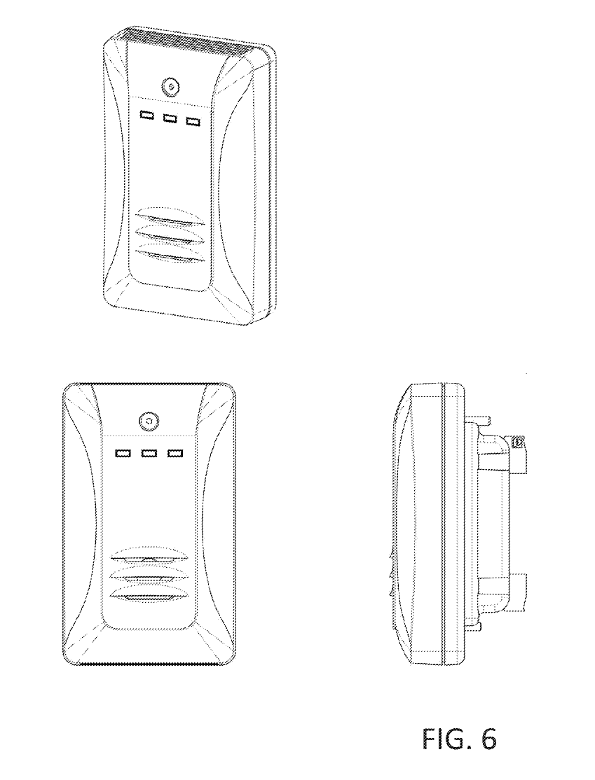

Referring to FIG. 6, an exemplary housing is illustrated to house the photo diode assembly, the sensor, optical elements, and a sensor processing unit. The output of the sensor may include an output signal, such as for example, a 0-5 volt signal, a 0-10 volt signal, a 4-20 ma signal, with or without a relay. The sensor processing unit typically includes a microprocessor for processing. In addition, light indicators may provide a signal indicative of current conditions, such as green for normal, yellow for low set point reached, and red for high set point reached.

In a preferred embodiment, the photo inversion diode included with the laser diode assembly typically includes a relatively small volume of gas operatively located between the output of the laser diode and the photo inversion diode. Preferably, this relatively small volume of gas is the same gas that is included within the enclosure which the laser diode assembly provides an optical output into. Accordingly, the laser diode assembly is preferably supported by the enclosure in such a manner that the gas within the enclosure is capable of also flowing to provide the relatively small volume of gas operatively located between the output of the laser diode and the photo inversion diode. Furthermore, having the same gas that is sensed within the enclosure to be the same as the gas operatively located between the output of the laser diode and the photo inversion diode, the temperature variations within the enclosure, and thus the temperature variations of the gas, will be the same (or substantially the same). In this manner, the feedback of the photo inversion diode will be affected in a similar manner to that of the photo-detector sensor.

In a preferred embodiment, the volume of the gas within the enclosure that is operatively located between the laser diode assembly and the photo-detector sensor is preferably greater than 50 times the relatively small volume of gas operatively located between the output of the laser diode and the photo inversion diode, and more preferably greater than 100 times the relatively small volume of gas operatively located between the output of the laser diode and the photo inversion diode. In this manner, the distortion that is a result of the relatively small volume of gas operatively located between the output of the laser diode and the photo inversion diode will be substantially smaller than the distortion that is a result of the gas within the enclosure that is operatively located between the laser diode assembly and the photo-detector sensor.

In a preferred embodiment, the class of material used in the photo inversion diode is the same class (or substantially the same class) of material used in the photo-detector sensor. For example, the material of the photo-inversion diode may be silicon, germanium, indium gallium arsenide, lead sulfide, and mercury cadmium telluride. Further, the material may be doped with different compounds. Further the material may be doped with different doping concentrations. In this manner, the photo inversion diode and the photo-detector sensor will respond in a substantially similar manner to temperature variations. With the gas of the photo-inversion detector and the photo-detector sensor being the same, the thermal environment of each of the detectors are the same, and likewise the thermal characteristics of the sensors substantially track one another.

In a preferred embodiment, the electrical bias (typically voltage) applied to the photo inversion diode and the photo detector sensor are substantially the same (e.g., reverse bias on a PIN diode). In this manner, the bias condition of each of the photo inversion diode and the photo detector sensor are the same, and thus provide substantially similar responses.

In other embodiments, depending on the nature of the sensor and the nature of the gas being sensed, a different wavelength or range of wavelengths of light may be used.

In other embodiments, depending on the nature of the gas desired to be sensed, the laser diode assembly may be modified to a different wavelength or range of wavelengths of light.

In other embodiments, depending on the desirable size of the housing the path of light may be generally direct from one side of the housing to the other.

In other embodiments, depending on the desirable size of the housing the path of light may be reflected, or otherwise directed in different directions with one or more lenses, to increase the path length before the light is sensed.

In other embodiments, the desirable size of the sensor may be modified, such as 1 square inch or more, 2 square inches or more, 12 square inches or more, etc.

In other embodiments, the sensor may be manufactured using other processes to provide a different range of sensitivities to light.

All the references cited herein are incorporated by reference.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

I/We claim:

1. A sensor for detecting a gas concentration comprising:
(a) a housing that defines at least one opening therein;
(b) a laser diode assembly supported by said housing that that provides an optical output that includes 405 nm wavelength of light directed within said housing;
(c) a photo detector sensor supported by said housing having an light sensitive area of at least 1 square inch positioned to sense said optical output;
(d) a sensor processing unit supported by said housing that receives an output from said photo detector sensor and estimates the level of a gas within said housing.

2. The sensor of claim 1 wherein said optical output is primarily 405±10 nm wavelength range.

3. The sensor of claim 2 wherein said optical output is 75% or more in the 405±10 nm wavelength range.

4. The sensor of claim 3 wherein said optical output is 90% or more in the 405±10 nm wavelength range.

5. The sensor of claim 1 wherein said laser diode assembly includes a laser diode that provides said optical output.

6. The sensor of claim 5 wherein said laser diode assembly includes a photo inversion diode.

7. The sensor of claim 6 wherein an output of said photo inversion diode is provided to said laser diode to modify the optical output of said laser diode.

8. The sensor of claim 7 wherein said modified optical output maintains a spectrum of said optical output substantially stable.

9. The sensor of claim 1 wherein said optical output is a cone of light.

10. The sensor of claim 9 wherein said optical output passes through at least one lens supported by said housing prior to being said received by said photo detector sensor.

11. The sensor of claim 9 wherein said optical output is reflected by at least one reflector supported by said housing prior to being said received by said photo detector sensor.

12. The sensor of claim 1 wherein said estimation of said level of said gas within said housing is based upon an attention of said optical output.

13. The sensor of claim 1 wherein said optical output of light is substantially 8 degrees from parallel and 21 degrees from perpendicular.

14. The sensor of claim 1 wherein said photo detector sensor includes a photovoltaic cell.

15. The sensor of claim 14 wherein said photo detector includes a silicon photovoltaic cell.

16. The sensor of claim 15 wherein said photo detector includes a single junction silicon photovoltaic cell.

17. The sensor of claim 1 wherein said photo detector undergoes insubstantial changes in its surface area based upon changes in ambient temperature between −5 degrees C. to 35 degrees C.

18. The sensor of claim 1 wherein said photo detector undergoes insubstantial changes in its surface area based upon changes in ambient pressure between 97 kilopascals to 103 kilopascals.

19. The sensor of claim 1 wherein said photo detector has said light sensitive area of at least 3 square inches.

20. The sensor of claim 1 wherein said photo detector has said light sensitive area of at least 6 square inches.

21. The sensor of claim 1 wherein said gas is NO2.

22. A sensor for detecting a gas concentration comprising:
(a) a housing that defines at least one opening therein;
(b) a laser diode assembly supported by said housing that that provides an optical output that includes an output wavelength of light in the range of 395 nm to 415 nm wavelength of light directed within said housing;
(c) a photo detector sensor supported by said housing having an light sensitive area of at least 1 square inch positioned to sense said optical output;
(d) a sensor processing unit supported by said housing that receives an output from said photo detector sensor and estimates the level of a gas within said housing.

23. The sensor of claim 22 wherein said optical output is primarily 405±10 nm wavelength range.

24. The sensor of claim 23 wherein said optical output is 75% or more in the 405±10 nm wavelength range.

25. The sensor of claim 24 wherein said optical output is 90% or more in the 405±10 nm wavelength range.

26. The sensor of claim 22 wherein said laser diode assembly includes a laser diode that provides said optical output.

27. The sensor of claim 26 wherein said laser diode assembly includes a photo inversion diode.

28. The sensor of claim 27 wherein an output of said photo inversion diode is provided to said laser diode to modify the optical output of said laser diode.

29. The sensor of claim 28 wherein said modified optical output maintains a spectrum of said optical output substantially stable.

30. The sensor of claim 22 wherein said optical output passes through at least one lens supported by said housing prior to being said received by said photo detector sensor.

31. The sensor of claim 22 wherein said optical output is reflected by at least one reflector supported by said housing prior to being said received by said photo detector sensor.

32. The sensor of claim 22 wherein said estimation of said level of said gas within said housing is based upon an attention of said optical output.

33. The sensor of claim 22 wherein said optical output of light is substantially 8 degrees from parallel and 21 degrees from perpendicular.

34. The sensor of claim 22 wherein said photo detector sensor includes a photovoltaic cell.

35. The sensor of claim 22 wherein said photo detector undergoes insubstantial changes in its surface area based upon changes in ambient temperature between −5 degrees C. to 35 degrees C.

36. The sensor of claim 22 wherein said photo detector undergoes insubstantial changes in its surface area based upon changes in ambient pressure between 97 kilopascals to 103 kilopascals.

37. The sensor of claim 22 wherein said photo detector has said light sensitive area of at least 3 square inches.

38. The sensor of claim 22 wherein said photo detector has said light sensitive area of at least 6 square inches.

39. The sensor of claim 22 wherein said photo detector has said light sensitive area of at least 2 square inches.

40. The sensor of claim 1 wherein said gas is NO2.

41. The sensor of claim 22 wherein a housing volume of gas is contained within said housing is operably located between said optical output and said photo detector that is greater than 50 times a laser diode assembly volume of gas operatively located between an output of said laser diode assembly and an input to an inversion diode of said laser diode assembly.

42. The sensor of claim 1 wherein a housing volume of gas is contained within said housing is operably located between said optical output and said photo detector that is greater than 50 times a laser diode assembly volume of gas operatively located between an output of said laser diode assembly and an input to an inversion diode of said laser diode assembly.
